(19) United States
(12) Patent Application Publication
(10) Pub. No.: US 2012/0215061 A1
(43) Pub. Date: Aug. 23, 2012

(54) HYDRAULIC GASTRIC BAND WITH REVERSIBLE SELF-OPENING MECHANISM

(75) Inventors: Pierre FRIDEZ, Froideville (CH); Tiago Bertolote, Geneva (CH); Xavier Raemy, Belmont-sur-Lausanne (CH); Fabian Kaegi, Lausanne (CH); Julien Rion, Morges (CH)

(73) Assignee: ALLERGAN, INC., Irvine, CA (US)

(21) Appl. No.: 13/029,409

(22) Filed: Feb. 17, 2011

(51) Int. Cl. A61F 2/04

(52) U.S. Cl. 600/37

(57) ABSTRACT

The present invention provides for a gastric banding system including an adjustment system that automatically increases the size of a constricted portion of a gastric lumen in response to an obstruction passing through the gastric lumen. The size of the constricted portion of the gastric lumen increases rapidly when the obstruction approaches a gastric band constricting the lumen. The adjustment system is also configured to slowly return the constricted gastric lumen to an equilibrium size the lumen had prior to the obstruction passing through the lumen. Various embodiments of gastric banding systems and adjustment systems are disclosed.

### FIELD

The present invention generally relates to medical systems and apparatus and uses thereof for treating obesity and/or obesity-related diseases, and more specifically, relates to gastric banding systems that self-adjust when an obstruction is present in a gastric lumen of a stomach of a patient.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts the food's passage into a lower portion of the stomach. When the stoma is of an appropriate size that is restricted by a gastric band, the food held in the upper portion of the stomach may provide a feeling of satiety or fullness that discourages overeating. Unlike gastric bypass procedures, gastric band apparatuses are reversible and require no permanent modification to the gastrointestinal tract. An example of a gastric banding system is disclosed in Roslin, et al., U.S. Patent Pub. No. 2006/0235448, the entire disclosure of which is incorporated herein by this specific reference.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

Birk, et al., U.S. application Ser. No. 12/816,310 discloses a hydraulic mechanical gastric band that includes an external control unit capable of communicating with a sensor to regulate the constriction of the band about the organ or the duct. The sensor sends data to the external control unit to control operations of the gastric band based on the data from the sensor. However, this application includes an external control unit to control operations of the gastric band.

Sometimes, adjustment of a gastric band may be desirable in between adjustments made by a physician. For example, during normal operation of the gastric band, the gastric band applies pressure to an outer surface of the upper stomach. But in some instances, the patient may swallow a bolus, or attempt to pass an obstruction (e.g., a large piece of food), that is too large to pass through the constriction produced by the gastric band. The result can be a painful experience which, if it persists, may require medical intervention to release the blockage.

Some attempts have been made to account for the possibility of a blockage. For example, Coe, et al., U.S. Patent Pub. No. 2009/0216255 discloses a flow control device that moves fluid between a hydraulic restriction system and a fluid source. The additional flow control device controls a rate of fluid flow between the restriction device and the fluid source. In addition, Coe, et al., European Patent Application No. 2 074 970 A1 discloses a separate restriction device and a pressure adjustment device. The pressure adjustment device regulates a constant force applied by the restriction device using, for example, a bellows and a spring.

Further, Lechner, U.S. Patent Pub. No. 2009/0054914 discloses a controllable stomach band that has a chamber for controlling restriction of the stomach band. The chamber is coupled to a separate pressure chamber that receives fluid leaving the chamber in the stomach band. The pressure chamber is separated from the esophageal-gastric junction of the patient's stomach.

Further, Steffen, U.S. Patent Pub. No. 2009/0062826 discloses an adjustable gastric band with a “conveyance device” that is powered by a “power storage device.” The power storage device operates the conveyance device to move fluid between expandable chambers to adjust the gastric band.

Some attempts have been made to account for the possibility of blockage (e.g., by a bolus of food). For example, Snow, et al., U.S. application Ser. No. 12/770,617 discloses a self-adjusting gastric band that temporarily and automatically opens up to allow a bolus through. However, this application does not utilize complicated fluid control mechanisms, flow rate limiting devices, and/or valves to regulate the transfer of fluid within the self-adjusting gastric band.

### SUMMARY

Accordingly, it is desirable to develop a gastric banding system that is capable of providing needed pressure to the stomach, yet is also capable of adapting and opening up to allow an obstruction to pass through a portion of the stomach being constricted. A reversible self-opening mechanism, or adjustment system, may be utilized to allow an obstruction to pass through a portion of a gastric lumen constricted by a gastric band. The adjustment system may allow the gastric band to open quickly in response to the obstruction passing through the gastric lumen, yet may also allow the gastric band to slowly return to the size the gastric band had before the obstruction was present.

In one embodiment, the present invention comprises a system including a gastric band having an inflatable member configured to contain fluid and apply constriction to a portion of a gastric lumen of the stomach, the inflatable member being moveable from a constricted state to a passage state for allowing an obstruction to pass through a portion of the gastric lumen, the passage state being when less fluid is contained in the inflatable member than in the constricted state. A valve is configured to move from a closed position to an open position when a pressure of the fluid from the inflatable member increases over a threshold in response to the obstruction passing through the gastric lumen, and to allow the fluid from the inflatable member to pass through the valve at a first flow rate. A reservoir is configured to receive the fluid passed through the valve, allowing the inflatable member to move from the constricted state to the passage state, and allowing the obstruction to pass through the portion of the gastric lumen. A flow restriction device is configured to allow the fluid received by the reservoir to pass from the reservoir to the inflatable member at a second flow rate that is less than the first flow rate, allowing the inflatable member to return to the constricted state.

In one embodiment, the valve and the flow restriction device are contained within an access port housing. A reservoir is coupled to the access port housing. The reservoir is configured to receive fluid from the gastric band automatically when an obstruction passes through the gastric lumen. The obstruction creates a force that is large enough to open the valve, which causes fluid to pass from the gastric band to the reservoir at a faster flow rate. Once the obstruction has passed through the constricted portion of the gastric lumen, the pressure of the reservoir is greater than that of the gastric band, and fluid then flows back to the gastric band through the flow restriction device, at a slower flow rate. The flow restriction device allows the gastric band to slowly return to an equilibrium size, or the size the gastric band had before the obstruction was present. The slow return of the gastric band to its equilibrium size prevents wear on the gastric band, and prevents damage to local tissues. The threshold opening pressure of the valve allows the gastric band to substantially maintain its size up to the threshold pressure, enhancing the therapeutic success of the gastric band.

In one embodiment, the present invention comprises a system including a gastric band having an inflatable member configured to contain fluid and apply constriction to a portion of a gastric lumen of the stomach, the inflatable member being moveable from a constricted state to a passage state for allowing an obstruction to pass through a portion of the gastric lumen, the passage state being when less fluid is contained in the inflatable member than in the constricted state. A valve is configured to move from a closed position to an open position when a pressure of the fluid in the inflatable member increases over a threshold in response to the obstruction passing through the gastric lumen, the valve in the open position allowing fluid to pass through the valve at a first flow rate. A reservoir is configured to receive fluid when the valve is in the open position, causing the inflatable member to move from the constricted state to the passage state, and allowing the obstruction to pass through the portion of the gastric lumen. A flow restriction device is configured to pass fluid through the flow restriction device at a second flow rate that is less than the first flow rate, allowing the inflatable member to return to the constricted state from the passage state.

In one embodiment, a shunt valve is incorporated into the gastric banding system. The shunt valve allows a physician to vary an amount of fluid in the gastric banding system without having to pass fluid through the flow restriction device.

In one embodiment, an asymmetric flow regulator, including a valve that is configured to open to allow fluid from the gastric band to pass through the valve, when a pressure of the fluid exceeds a threshold, and a flow restriction device, is utilized. The asymmetric flow regulator is positioned in series with a reservoir and an access port.

In one embodiment, an asymmetric flow regulator, including a valve that is configured to open to allow fluid from the gastric band to pass through the valve, when a pressure of the fluid exceeds a threshold, and a flow restriction device, is utilized. The asymmetric flow regulator is positioned in parallel with a reservoir and an access port.

### BRIEF DESCRIPTION OF THE DRAWINGS

FIG. 1 illustrates a perspective view of a gastric banding system according to an embodiment of the present invention.

FIG. 2 illustrates a perspective view of a gastric band according to an embodiment of the present invention.

FIG. 3 illustrates a perspective view of a gastric band according to an embodiment of the present invention.

FIG. 4 illustrates a perspective view of a gastric banding system according to an embodiment of the present invention.

FIG. 5A illustrates a schematic view of a gastric banding system according to an embodiment of the present invention.

FIG. 5B illustrates a schematic view of the gastric banding system in the state shown in FIG. 5A according to an embodiment of the present invention.

FIG. 6A illustrates a schematic view of a gastric banding system according to an embodiment of the present invention.

FIG. 6B illustrates a schematic view of the gastric banding system in the state shown in FIG. 6A according to an embodiment of the present invention.

FIG. 7A illustrates a schematic view of a gastric banding system according to an embodiment of the present invention.

FIG. 7B illustrates a schematic view of the gastric banding system in the state shown in FIG. 7A according to an embodiment of the present invention.

FIG. 8A illustrates a schematic view of a gastric banding system according to an embodiment of the present invention.

FIG. 8B illustrates a schematic view of the gastric banding system in the state shown in FIG. 8A according to an embodiment of the present invention.

FIG. 9 illustrates a side cross-section view of an access port according to an embodiment of the present invention.

FIG. 10 illustrates a side cross-section view of an access port according to an embodiment of the present invention.

FIG. 11 illustrates a perspective view of a gastric banding system according to an embodiment of the present invention.

FIG. 12 illustrates a schematic view of the gastric banding system shown in FIG. 11 according to an embodiment of the present invention.

FIG. 13 illustrates a perspective view of a gastric banding system according to an embodiment of the present invention.

FIG. 14 illustrates a schematic view of the gastric banding system shown in FIG. 13 according to an embodiment of the present invention.

### DETAILED DESCRIPTION

The present invention generally provides for gastric banding systems, for example, systems for the treatment of obesity and obesity related conditions, as well as systems that allow adjustment of gastric bands in response to an obstruction passing through a gastric lumen.

The present invention provides for an adjustment system that automatically increases the size of a constricted portion of a gastric lumen in response to an obstruction passing through the gastric lumen. The size of the constricted portion of the gastric lumen increases rapidly when the obstruction approaches a gastric band that constricts the lumen. The adjustment system is also configured to slowly return the constricted gastric lumen to an equilibrium size the lumen had, prior to the obstruction passing through the lumen.

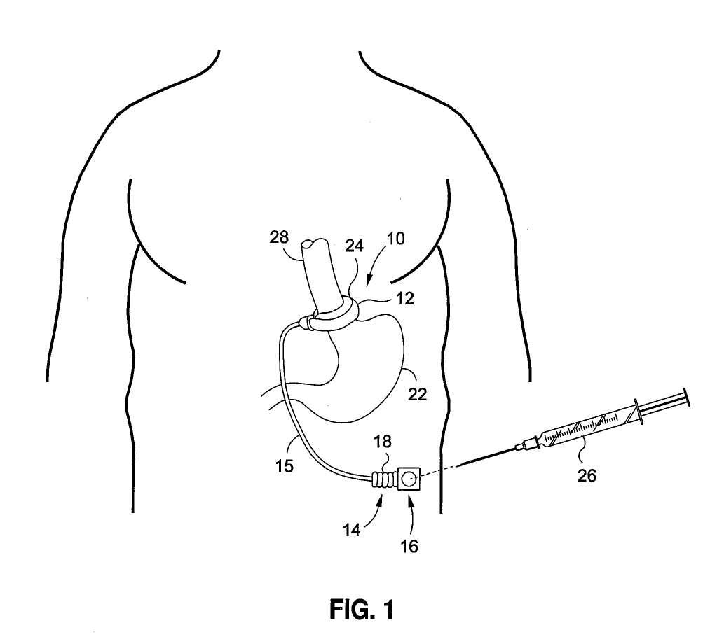

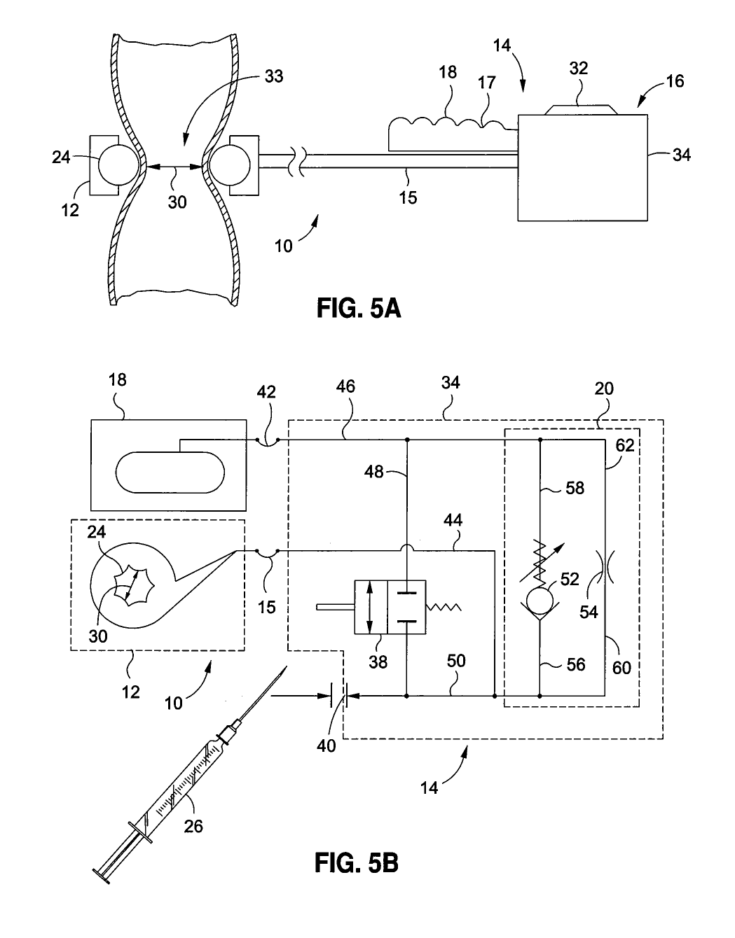

FIG. 1 illustrates an embodiment of the present invention including a gastric banding system 10 comprising a gastric band 12, an adjustment system 14, and a tube 15 connecting the gastric band 12 to the adjustment system 14. In the embodiment shown in FIG. 1, the adjustment system 14 includes an access port 16, a reservoir 18, and an asymmetric flow regulator 20 (visible in FIG. 5B) contained within the access port 16.

The gastric band 12 comprises a strap-like member capable of encircling a portion of a patient's stomach 22 to form a stoma. The gastric band 12 is preferably a hydraulic gastric band, having an inflatable member 24 that is filled with a fluid, such as saline. The inflatable member 24 serves as a cuff or a ring around a portion of the patient's stomach, which constricts the stomach, to a degree, based on the amount of fluid in the inflatable member 24.

The access port 16 comprises an implantable device that is used by a physician to inflate the inflatable member 24 of the gastric band 12. The access port 16 is configured to be fixed subcutaneously within the patient's body. The access port 16 is preferably fixed to the patient's muscle wall. The access port 16 is fixed to the muscle wall through sutures, anchors, tacks, or the like. The access port 16 is capable of receiving a syringe 26 that is inserted by a physician to transfer fluid to and from the inflatable member 24 of the gastric band 12. The fluid passes between the access port 16 and the inflatable member 24 through the tube 15.

The gastric band 12 is inserted into the patient's body laparoscopically. During laparoscopic insertion of the gastric band 12, the gastric band 12 is wrapped around the portion of the patient's body to be constricted, and is then secured in position. The inflatable member 24 contacts the portion of the patient's stomach to be constricted. The gastric band 12 preferably encircles the cardia, or esophageal junction, of the patient's stomach. After the gastric band 12 has been fixed around a portion of the patient's stomach, the access port 16 is fixed to the patient's muscle wall. The tube 15 is then connected from the inflatable member 24 of the gastric band 12 to the access port 16. A physician will then inject the access port 16 with an amount of fluid, as desired, to inflate the inflatable member 24 to an appropriate degree or size, depending on the physical characteristics of the patient, and the desired treatment plan for the patient. For example, if the patient is severely obese, then a greater degree of restriction and a greater amount of fluid may be necessary to constrict the portion of the patient's stomach. If the patient is mildly obese, then a lesser degree of restriction, and less fluid may be passed to the inflatable member 24, than if the patient is severely obese.

The patient's stomach is constricted in order to treat obesity. The constriction causes the food to pass from the patient's esophagus 28 to the lower portions of the patient's stomach 22, at a rate that is slower than would normally occur without the restriction. The decreased rate of food flow increases a feeling of fullness for the patient, and enhances satiety signals that are sent to the patient's brain. The enhanced feeling of fullness causes the patient to reduce food consumption, which desirably causes the patient to lose weight.

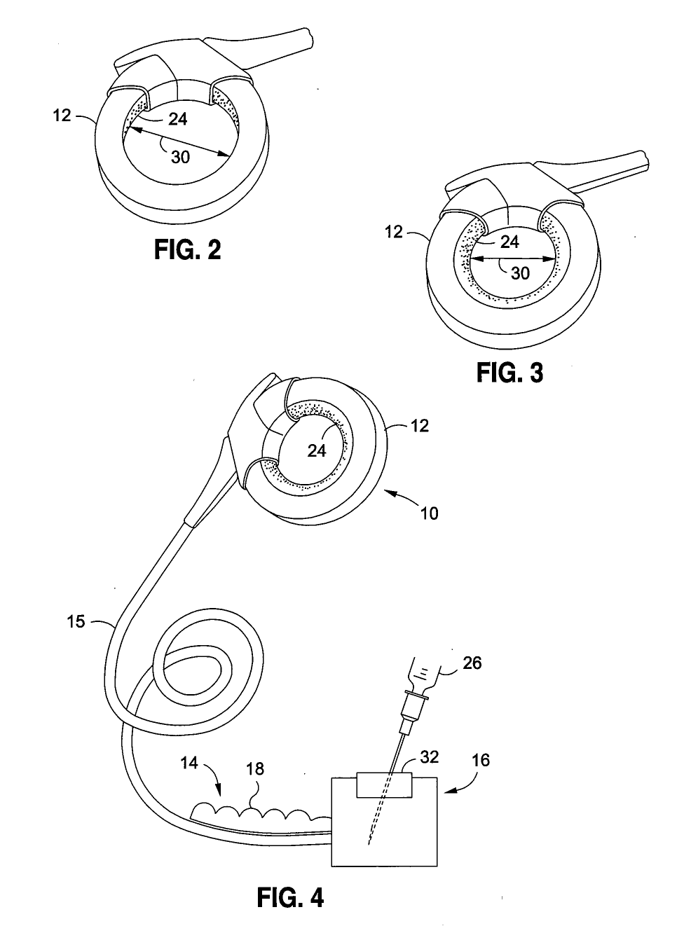

FIG. 2 illustrates a perspective view of the gastric band 12 and the inflatable member 24 of FIG. 1. The gastric band 12 is designed to form a loop around the patient's stomach, and form an inner diameter 30 which defines the size of a constriction of the patient's stomach. If the inner diameter 30 size is reduced, then the restriction of the patient's stomach increases. The size of the constriction, or constricted portion of the patient's stomach, accordingly decreases.

FIG. 3 illustrates a perspective view of the gastric band 12 with an increased amount of fluid passed into the inflatable member 24. The diameter 30 size is reduced, causing an increased restriction of the patient's stomach, and accordingly, a decreased size of the constriction.

FIG. 4 illustrates a perspective view of the gastric banding system 10 of FIG. 1, including the gastric band 12 and the adjustment system 14. The syringe 26 is illustrated to penetrate a septum 32 of the access port 16, in a position that would allow a physician to vary an amount of fluid in the inflatable member 24.

FIG. 5A illustrates a schematic representation of the constriction of a portion of the patient's stomach formed by the gastric band 12. The inflatable member 24 encircles a portion of the patient's stomach to form a constriction. The diameter 30 of the gastric band defines a size of a gastric lumen 33, or interior cavity of the patient's stomach, for food to pass through, from the patient's esophagus to the lower portions of the patient's stomach. The size, or cross-sectional surface area, of the lumen 33 defines the size of the passageway through which food can pass. If the lumen 33 is small, then food can not easily pass through the lumen 33. If the lumen 33 is large, then food can more easily pass through the lumen 33.

In a standard gastric banding system, the size of the constriction of the patient's stomach, and the size of the gastric lumen 33, is fixed. In other words, in a standard gastric banding system, after the gastric band and the access port have been implanted into a patient's body, the size of the lumen 33 is set by the physician, via the access port. In one embodiment, the size of the lumen 33 may only be adjusted if the physician inserts a syringe into the access port and adds or removes fluid from the gastric band lumen 33. However, a lumen 33 with a fixed size may be undesirable if an obstruction, or a large bolus of food, attempts to pass through the lumen 33. The obstruction may become stuck in the lumen 33 as it may not be able to pass through the construction formed by the gastric band. This result is undesirable because it could cause pain for the patient. The patient may need to visit a physician to loosen the band in order to allow the obstruction to pass through the constriction. The obstruction may comprise a large bolus of food traveling through the esophagus to the patient's stomach. The obstruction may also comprise the patient's vomit, attempting to travel from the patient's stomach up through the patient's esophagus.

It is thus desirable if an adjustment system 14 is incorporated into the gastric banding system 10, to allow a size of a portion of the gastric lumen 33 constricted by a gastric band to vary in response to an obstruction passing through the gastric lumen 33.

The components and operation of the adjustment system 14 will now be discussed in relation to FIGS. 5A and 5B. As shown in FIG. 5A, and as discussed in relation to FIG. 1, the adjustment system 14 includes an access port 16, a reservoir 18, and an asymmetric flow regulator 20 (visible in FIG. 5B).

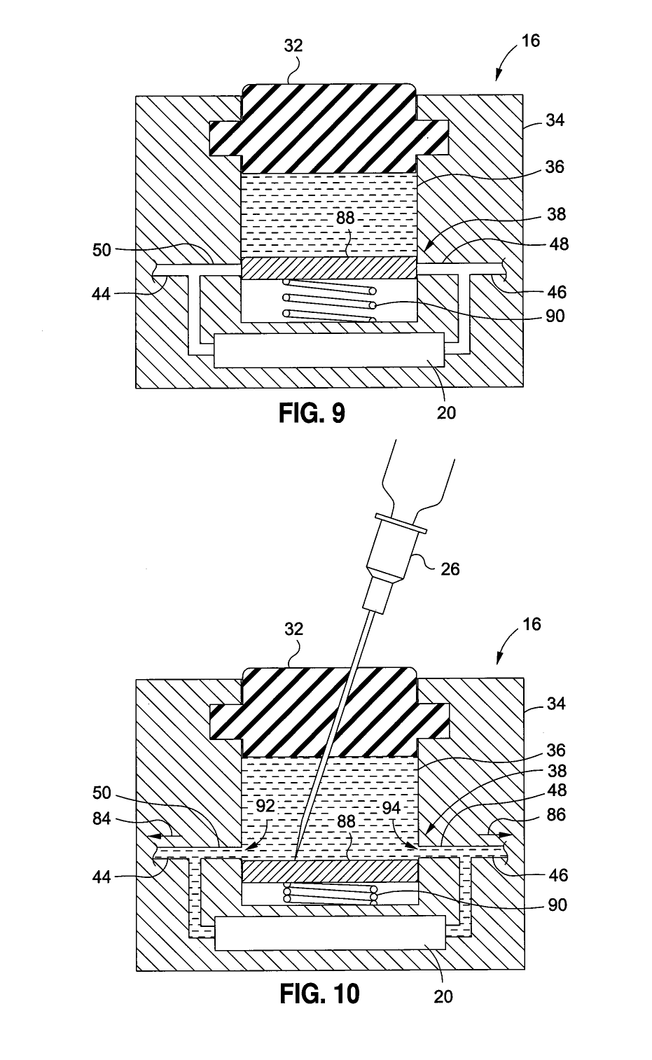

The access port 16 includes a housing 34, a septum 32 and a fluid chamber 36 (visible in FIGS. 9 and 10). The housing 34 forms an outer shell of the access port 16, and retains the septum 32 and the fluid chamber 36. The housing 34 is configured to be biocompatible, to allow the housing 34 to integrate biologically with the local tissues positioned around the housing 34. The septum 32 is configured to allow a syringe needle to pass through the septum 32 and enter the fluid chamber 36, to add or remove fluid from the gastric band 12. The septum 32 prevents fluid from exiting the fluid chamber 36 when the syringe needle penetrates through the septum 32, and after the syringe needle is withdrawn from the septum 32.

The reservoir 18 comprises a structure configured to receive fluid from the gastric band 12. The reservoir 18 may comprise a flexible, or elastic, structure having a volume capable of automatically varying to accommodate an amount of fluid entering the reservoir 18. In the embodiment shown in FIG. 5A, the reservoir 18 may comprise a flexible balloon or bladder structure, capable of increasing in size in response to an amount of fluid entering the reservoir 18. The reservoir 18 may stretch to accommodate fluid entering the reservoir 18, and may shrink to accommodate fluid exiting the reservoir 18. In addition, as shown in FIG. 5A, the reservoir 18 may include a ridged structure 17, including a plurality of notches on the surface of the reservoir 18. The notches enhance the flexibility of the reservoir 18 by forming pre-stressed regions of the reservoir 18.

The physical properties of the reservoir 18 may be selected as desired to effect various performance attributes of the adjustment system 14. For example, a relatively large reservoir 18 may allow the reservoir 18 to more quickly receive fluid from the gastric band 12. A larger reservoir 18 will allow a large quantity of fluid to pass from the gastric band 12 to the reservoir 18. In addition, a more flexible reservoir 18 may allow fluid to more quickly pass to the reservoir 18 from the gastric band 12. Further, a smaller reservoir 18 may reduce the total size of the adjustment system 14. Moreover, a less flexible reservoir 18 may reduce the disruption caused by the reservoir 18 towards local tissues, when the reservoir 18 inflates with fluid. The reservoir 18 may be made of an elastic resilient material, such as silicone, or may be made of a rubber or appropriate plastic. The materials selected to form the reservoir 18 may be varied as desired, and the size of the reservoir 18 may be varied as desired. In one embodiment, the reservoir 18 may be sized such that the gastric banding system 10 contains approximately 56 mL (fifty-six milliliters) of fluid. This volume is exemplary and may be varied as desired.

The reservoir 18 is coupled to the housing 34 through a coupling device, namely, through a tube coupler or a connecting nipple structure. Other appropriate coupling devices may be utilized as desired. The reservoir 18 may be securely or detachably fixed to the housing 34. In an embodiment in which the reservoir 18 is detachably fixed to the housing 34, a detachable locking mechanism may be used to couple the reservoir 18 to the housing 34.

The reservoir 18 is preferably positioned exterior to the housing 34 to accommodate size changes of the reservoir 18. In an embodiment in which the reservoir 18 comprises a bladder or balloon configured to vary in size, the externally placed reservoir 18 may allow the reservoir 18 to expand without being confined by the size of the housing 34. In one embodiment, the reservoir 18 may be positioned external to the housing 34, yet placed within a shell that is configured to house the reservoir 18. The shell may be sized to allow the reservoir 18 to increase or decrease in size without interference from the interior surfaces of the shell (e.g., restrained by the interior surfaces of the shell).

Further, the reservoir 18 is preferably positioned exterior to the housing 34, to allow the reservoir 18 to be removed from the housing 34 and replaced as necessary. As discussed above, the physical properties of the reservoir 18 may be varied as desired. A physician may determine a larger reservoir 18 is necessary for use in the adjustment system 14. An external reservoir may more easily allow a physician to access the reservoir 18 and replace the reservoir 18, without having to remove the gastric band 12 or the access port 16 from the patient's body.

FIG. 5A illustrates the reservoir 18 extending along the length of the tube 15, and extending in a direction towards the gastric band 12. The position of the reservoir 18 along the tube 15 allows the profile of the adjustment system 14 to be reduced, as the tissue displaced by the reservoir 18 has already been displaced by the path of the tube 15 leading towards the gastric band 12. Further, FIG. 5A illustrates the reservoir 18 positioned above the tube 15. The position of the reservoir 18 above the tube 15 allows the physician to more easily access the reservoir 18 without interference from the tube 15 extending from the housing 34.

FIG. 5B illustrates a schematic view of the gastric banding system 10 shown in FIG. 5A. The gastric banding system 10 includes the gastric band 12, schematically outlined in a dashed box, and the adjustment system 14. The adjustment system 14 includes the reservoir 18, schematically outlined in a solid box, and the asymmetric flow regulator 20, schematically outlined in a dashed box. The access port housing 34 is schematically represented by a dashed box. The asymmetric flow regulator 20 and a shunt valve 38 are shown positioned within the access port housing 34. A schematic representation of a filling point 40 is also shown, positioned within the access port housing 34.

The tube 15 connects the gastric band 12 to the access port housing 34. A fluid conduit 44 connects the tube 15 to the asymmetric flow regulator 20. A coupler 42 connects the reservoir 18 to the access port housing 34. A fluid conduit 46 connects the asymmetric flow regulator 20 to the coupler 42. A fluid conduit 48 connects the shunt valve 38 to the conduit 46 leading to the coupler 42. A fluid conduit 50 connects the shunt valve 38 to the conduit 44 leading to the tube 15.

The asymmetric flow regulator 20 comprises a mechanism including a valve that is configured to open to allow fluid from the gastric band 12 to pass through the valve, when a pressure of incident fluid exceeds a threshold. The asymmetric flow regulator 20 further comprises a flow restriction device that is configured to allow fluid from the reservoir 18 to pass to the gastric band 12. In the embodiment shown in FIG. 5B, the valve is configured as a check valve, or a one-way valve 52, that only allows flow in one direction, and opens when an incident fluid pressure exceeds a threshold pressure.

In the embodiment shown in FIG. 5B, the one-way valve 52 comprises a variable resistance ball check valve, or a check valve that prevents fluid flow in a first direction by the force of a spring pressing against a ball. The one-way valve 52 is moveable from a closed position, in which no flow is allowed, to an open position, in which flow is allowed through the one-way valve 52. The one-way valve 52 only allows flow in the opposite or second direction, to the first direction, when a pressure of incident fluid exceeds a defined threshold value. The one-way valve 52 thus only allows flow in the direction of: from the inflatable member 24 to the reservoir 18. The one-way valve 52 opens automatically when the pressure of incident fluid exceeds a defined threshold value. The threshold value is defined by the degree of force the spring exerts against the ball.

The threshold value of the one-way valve 52 can be adjusted by varying the compressive force of the spring against the ball, as desired. The compressive force may be varied either mechanically, or hydraulically, as is known in the art. For example, a twisting mechanism may vary the spring constant of the spring, and consequently increase the compressive force of the spring. In addition, a hydraulic pressure may press against the spring to vary the compressive force of the spring. The threshold value of the one-way valve 52 may be adjusted either prior to implantation of the housing 34 or after the housing 34 has been implanted into the patient's body. In one embodiment, the physician may insert a syringe into the housing 34, after the housing 34 has been implanted, to hydraulically vary the threshold value of the one-way valve 52. The threshold value is defined by a structural configuration of the one-way valve 52, or, in other words, a physical property of the one-way valve 52 (e.g., the tension of the spring) defines the value of the threshold force required to open the valve 52.

In one embodiment, the one-way valve 52 may be configured as a valve such as an adjustable diaphragm valve, or adjustable duckbill valve. In one embodiment, the one-way valve 52 may be adjusted through telemetric means, in which a physician wirelessly communicates with a controller that controls the opening and closing of the one-way valve 52, and the threshold opening pressure of the one-way valve 52. In one embodiment, the one-way valve 52 may be configured to comprise any equivalent mechanism capable of only allowing fluid flow in one direction. In one embodiment, the valve of the asymmetric flow regulator 20, shown in FIG. 5B, to comprise a one-way valve 52, may comprise any mechanism capable of opening to allow fluid flow when incident fluid pressure is above a threshold pressure value.

In the embodiment shown in FIG. 5B, the threshold value at which the one-way valve 52 may allow fluid flow, may be set as a pressure differential across the one-way valve 52. In other words, the one-way valve 52 may open when the pressure of fluid incident from the inflatable member 24 of the gastric band 12 is greater than the pressure of fluid contained within the reservoir 18. For example, a pressure differential of 10 mmHg (ten millimeters of mercury) across the one-way valve 52 may cause the one-way valve 52 to open. This threshold value is exemplary, and may be varied as desired. In one embodiment, the threshold value may be an absolute value, of which the one-way valve 52 opens when the pressure of fluid incident from the inflatable member 24 raises above this value. The absolute pressure value may be, for example, 20 mmHg. The absolute pressure value may also be in a range of between approximately 10 mmHg to 80 mmHg. This threshold value is exemplary, and may be varied as desired.

The asymmetric flow regulator 20 preferably includes a flow restriction device, shown in FIG. 5B, to comprise a flow control valve 54. The flow control valve 54 preferably comprises a device that reduces the flow rate of fluid passing through the flow control valve 54. The flow control valve 54 may comprise a narrow fluid conduit that includes an obstruction in the flow path of the conduit. The flow rate through the flow control valve 54 may be reduced by narrowing the flow path for fluid passing through the flow control valve 54, by reducing the cross-sectional area for the fluid to pass through by placing the obstruction in the flow path, to impede the passage of the fluid through the flow control valve 54. In one embodiment, the flow rate may be defined as a volume of fluid passing through the flow control valve 54 per unit time.

In one embodiment, the flow control valve 54 may comprise a variable flow control valve 54, capable of varying the flow rate passing through the flow control valve 54. For example, the flow control valve 54 may comprise a tube having inflatable walls that surround a fluid passageway. The rate of fluid passage through the walls depends on the degree to which the walls are inflated. The flow rate may therefore be varied by varying the amount of fluid contained within the walls of the valve 54, and therefore varying the size of the walls. The amount of fluid in the walls may be varied prior to, or after implantation of the housing 34. A physician may insert a syringe into the access port housing 34 to add or remove fluid from the walls of the valve 54.

In one embodiment, the flow restriction device may comprise a narrow fluid conduit, being sized more narrowly than other fluid conduits utilized in the adjustment system 14, for example, conduits 44, 46, 48, 50, or fluid conduits 56, 58, 60, 62. For example, in this embodiment, the flow restriction device may comprise a narrow tubing. This tubing may allow fluid to pass from the reservoir 18 to the gastric band 12 at a rate that is less than a rate at which fluid passes through the one-way valve 52.

In one embodiment, the flow restriction device may comprise any mechanism capable of reducing the flow rate of fluid passing therethrough. In one embodiment, the restricted flow rate through a flow restriction device may be defined as a rate, being less than the rate at which fluid is capable of flowing through the one-way valve 52, when the one-way valve 52 is open. In one embodiment, the restricted flow rate through a flow restriction device may be defined as a rate that causes the inflatable member 24 of the gastric band 12 to fill at a rate, which is less than the rate at which fluid exits the gastric band 12, when it passes through the one-way valve 52.

The various conduits 44, 46, 48, 50, 56, 58, 60, 62 shown schematically in FIG. 5B may be implemented as tubes, channels, or passageways. In one embodiment, the conduits 44, 46, 48, 50, 56, 58, 60, 62 may be formed as structural elements of the access port housing 34. In one embodiment, the conduits 44, 46, 48, 50, 56, 58, 60, 62 may be formed during the molding process that is used to form the access port housing 34.

In the embodiment shown in FIG. 5B, a fluid conduit 56 fluidly links the one-way valve 52 with the conduit 44 that fluidly communicates with the inflatable member 24. A fluid conduit 58 also fluidly links the one-way valve 52 with the conduit 46 that fluidly communicates with the reservoir 18. The fluid conduits 56, 58 also link the one-way valve 52 with the fluid conduits 60, 62 leading to the flow control valve 54. The one-way valve 52 is therefore in fluid communication with the inflatable member 24 of the gastric band 12, and with the flow control valve 54, and with the reservoir 18. The one-way valve 52 is also in fluid communication with the shunt valve 38.

In the embodiment shown in FIG. 5B, a fluid conduit 60 fluidly links the flow control valve 54 with the conduit 44 that fluidly communicates with the gastric band 12. A fluid conduit 62 fluidly links the flow control valve 54 with the conduit 46, which fluidly communicates with the reservoir 18. The fluid conduits 60, 62 also link the flow control valve 54 with the fluid conduits 56, 58 leading to the one-way valve 52. The flow control valve 54 is therefore in fluid communication with the inflatable member 24 of the gastric band 12, with the one-way valve 52, and with the reservoir 18. The flow control valve 54 is also in fluid communication with the shunt valve 38.

In the embodiment shown in FIG. 5B, the one-way valve 52 and the flow control valve 54 are in fluid communication with the inflatable member 24 of the gastric band 12 in a parallel configuration. In addition, the one-way valve 52 and the flow control valve 54 are in fluid communication with the reservoir 18 in a parallel configuration. The fluid may flow from the inflatable member 24 of the gastric band 12 through either the flow control valve 54, or through both the flow control valve 54 and the one-way valve 52, depending on the pressure of the fluid passing from the inflatable member 24. The one-way valve 52 is in direct fluid communication with the reservoir 18 and the inflatable member 24, because no valve or control device impedes fluid flow between the one-way valve 52 and the reservoir 18, and the one-way valve 52 and the inflatable member 24. The one-way valve 52 is also in direct fluid communication with the flow control valve 54, because no valve or control device impedes fluid flow between the one-way valve 52 and the flow control valve 54. The flow control valve 54 is in direct fluid communication with the reservoir 18 and the inflatable member 24, because no valve or control device impedes fluid flow between the flow control valve 54 and the reservoir 18, and the flow control valve 54 and the inflatable member 24.

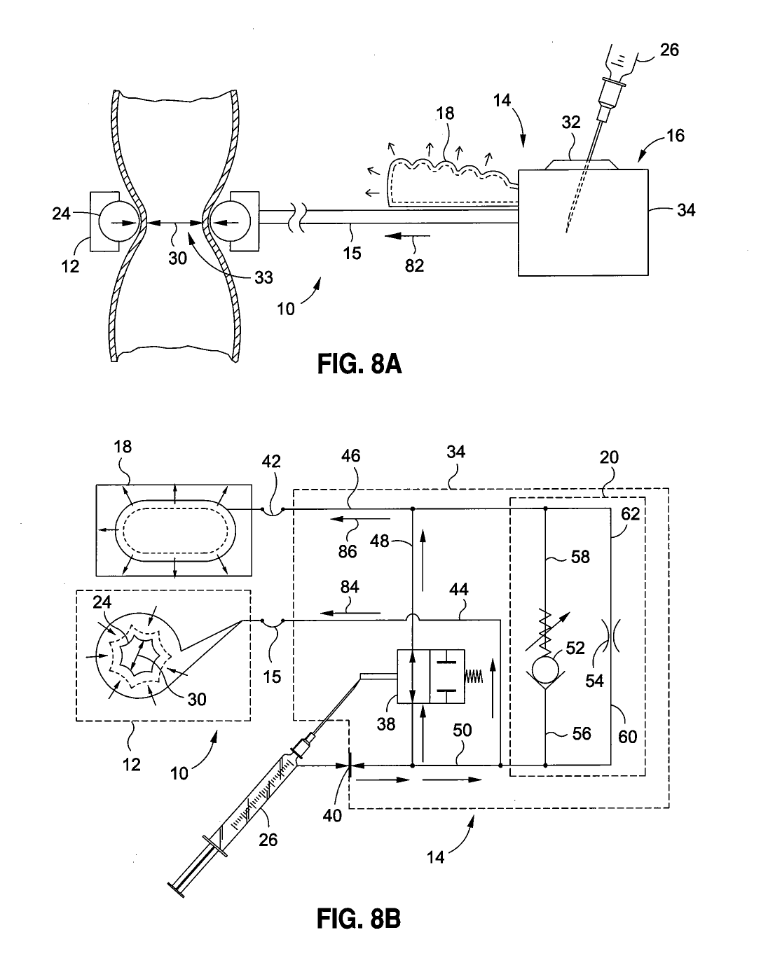

The shunt valve 38, to be discussed further in relation to FIGS. 9A and 9B, comprises a valve that is opened or closed at the selection of a user, who preferably comprises a physician. In the configurations of the gastric banding system 10 shown through FIGS. 5A through 7B, the shunt valve 38 remains closed, to prevent fluid from passing through the shunt valve 38. The shunt valve 38 is shown in an open configuration in FIGS. 8A and 8B.

Referring back to FIG. 5A, in operation, the adjustment system 14 automatically increases the size of the constricted portion of the gastric lumen 33 in response to an obstruction passing through the gastric lumen 33. The size of the constricted portion of the gastric lumen 33 increases rapidly when the obstruction approaches the gastric band 12. The fluid that fills the inflatable member 24 rapidly travels through the tube 15 and fills the reservoir 18. The decreased amount of fluid in the inflatable member 24 increases the diameter 30 of the gastric band 12, and increases a size of the gastric lumen 33.

The adjustment system 14 is also configured to slowly return the size of the constricted gastric lumen 33 to the size it had prior to the obstruction passing through the lumen 33, or the size when no obstruction is passing through.

FIGS. 5A and 5B illustrate the gastric banding system 10 in an equilibrium state, in which the gastric band 12 is in an equilibrium position. The equilibrium position may be equivalently referred to as the constricted state of the inflatable member 24. In the equilibrium state, the pressure of fluid within the inflatable member 24 is equal, or nearly equal to the pressure of fluid within the reservoir 18. The pressure of the inflatable member 24, or the pressure difference between the inflatable member 24 and the reservoir 18, is not sufficient to open the one-way valve 52. In this state, fluid may only pass from the inflatable member 24 to the reservoir 18 through the flow control valve 54.

The equilibrium pressure of the inflatable member 24 and the reservoir 18 is preferably set by a physician, to optimally reduce food intake for the patient, depending on the unique physical characteristics and size of the patient. For example, if the physician desires that a greater degree of constriction be applied to the gastric lumen 33, then a greater amount of fluid may be passed into the gastric banding system 10, which increases the equilibrium pressure. A physician may adjust the equilibrium pressure by varying the amount of fluid in the gastric banding system 10 through use of the shunt valve 38, in a process discussed more fully in relation to FIGS. 8A and 8B.

Forces may be exerted against the gastric band 12 that cause the equilibrium state to be disturbed. If the equilibrium state is disrupted by forces, to the extent that the one-way valve 52 does not open, then fluid will slowly pass through the flow control valve 54 between the reservoir 18 and the inflatable member 24, to maintain the equilibrium state, between the pressures of the inflatable member 24 and the reservoir 18. For example, the one-way valve's 52 threshold pressure may be set at a differential of 10 mmHg between the inflatable member 24 and the reservoir 18. Further, the pressure of the inflatable member 24 in an equilibrium state may be 11 mmHg, and the pressure of the reservoir 18 may be 11 mmHg. If forces are exerted against the gastric band 12 to raise the pressure of the inflatable member 24 to 15 mmHg, then the 10 mmHg differential has not been met. Fluid will then only flow through the flow control valve 54 at a slow flow rate, until the pressures of the inflatable member 24 and the reservoir 18 equalize at 15 mmHg. The size of the gastric lumen 33 and the size of the gastric band's 12 diameter 30 increase in response to the fluid being transferred from the inflatable member 24 to the reservoir 18.

The slow flow of fluid through the flow control device 54 beneficially maintains equilibrium across the inflatable member 24 and the reservoir 18, and adjusts a size of the gastric lumen 33 in response to prolonged forces exerted against the gastric band 12. The flow through the flow control device 54, and the adjusted size of the gastric lumen 33 occur until an equilibrium pressure is reached between the pressures of the inflatable member 24 and the reservoir 18.

The slow flow of fluid through the flow control valve 54, however, may not be sufficient to adjust a size of the gastric band 12 quickly, in response to an obstruction passing through the gastric lumen 33. An alternate mechanism, namely, the one-way valve 52, is utilized to allow fluid to pass at a large, or fast, flow rate from the inflatable member 24 to the reservoir 18, to quickly increase the size of the gastric lumen 33, and accordingly quickly increase the diameter 30 of the gastric band 12. The fast flow rate may be defined as a rate being faster than fluid may pass through the flow restriction device, represented in FIG. 5B as a flow control valve 54.

A large force exerted against the gastric band 12 will overcome the threshold pressure of the one-way valve 52. The one-way valve 52 will open in response to pressure of the fluid in the inflatable member 24 increasing over a threshold. The large force exerted against the gastric band 12 may be caused by an obstruction passing through the gastric lumen 33. Fluid will pass from the inflatable member 24 to the reservoir 18 at a fast flow rate through the one-way valve 52. Fluid will pass through the one-way valve 52 until the threshold pressure differential is met, at which time the one-way valve 52 will close. The remaining pressure differential may be equalized through fluid flow through the flow control valve 54.

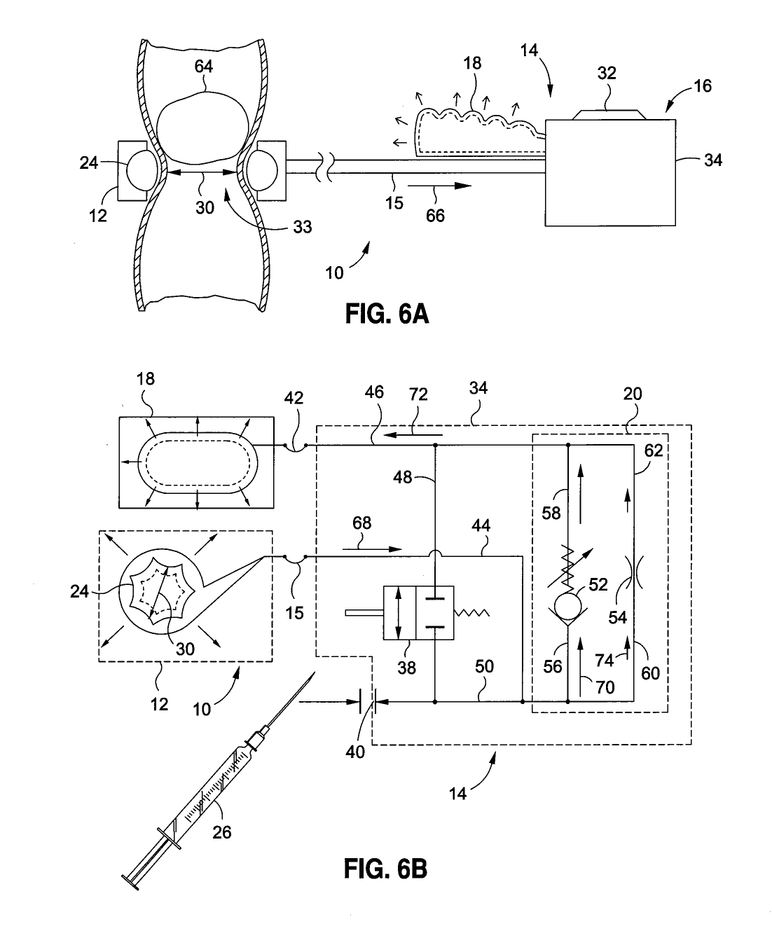

FIG. 6A illustrates a schematic representation of an obstruction 64 passing through the patient's gastric lumen 33. The obstruction 64 represents any of the embodiments of an obstruction discussed throughout this disclosure, including a large bolus of food. The obstruction 64 may also comprise any other object, which could not pass through the constriction formed by the gastric band 12, when the gastric band 12 is at its equilibrium position. In the embodiment shown in FIGS. 6A and 6B, if the obstruction 64 exerts a force against the gastric band 12 large enough to overcome the pressure threshold of the one-way valve 52, then the one-way valve 52 will open, and will allow fluid to pass from the inflatable member 24 to the reservoir 18 at a fast flow rate. FIG. 6A illustrates such a state of the gastric banding system 10, in which the force from the obstruction 64 against the gastric band 12 is sufficient to overcome the pressure threshold of the one-way valve 52.

As shown in FIG. 6A, a long arrow 66 representing fluid flow at a fast rate, indicates fluid passing from the inflatable member 24 to the reservoir 18. The size of the diameter 30 of the gastric band 12 increases, as the volume of fluid in the inflatable member 24 is reduced. The size of the reservoir 18 increases, as the fluid has passed from the inflatable member 24 to the reservoir 18.

FIG. 6B illustrates a schematic view of the gastric banding system 10 in the state shown in FIG. 6A. The diameter of the inflatable member 24 of the gastric band 12 increases. The inflatable member 24 moves to a passage state, or passage size, for allowing the obstruction 64 to pass through the gastric lumen 33. Less fluid is contained in the inflatable member 24 in the passage state than in the constricted state. The inflatable member 24 is moveable from the passage state, to the constricted state, and from the constricted state to the passage state.

A long arrow 68 indicates a fluid flow at a fast flow rate passing to the one-way valve 52. A long arrow 70 indicates fluid flow at a fast flow rate passing through the one-way valve 52. A long arrow 72 indicates a fluid flow at a fast flow rate passing to the reservoir 18. A short arrow 74 indicates a fluid flow at a slow flow rate through the flow control valve 54. The fast flow rate is greater than the slow flow rate.

FIG. 6B further illustrates the size of the reservoir 18 increasing as it fills with fluid. The reservoir 18 receives the fluid that is passed through the one-way valve 52, to allow a size of the portion of the gastric lumen 33 being constricted, to increase in response to the obstruction passing through the gastric lumen 33, to allow the obstruction to pass through the portion of the gastric lumen 33.

The adjustment system 14 beneficially rapidly increases the size of the portion of the gastric lumen 33 that is constricted in response to an obstruction 64 passing through the gastric lumen 33. The adjustment system 14 does so automatically, or without user intervention, in response to luminal 33 pressure increasing or rising above a threshold level. The rapid increase of the size of the portion of the gastric lumen 33 beneficially allows the obstruction 64 to quickly pass through the constriction of the gastric lumen 33. It is desired that the diameter 30 of the inflatable member 24 is able to increase to the passage state, or passage size, which allows the obstruction 64 to pass through the gastric lumen 33.

The flow of fluid from the inflatable member 24 to the reservoir 18 occurs until the pressure of the reservoir 18 equals or is approximately equal to the pressure of the inflatable member 24.

The force exerted by the obstruction 64 against the gastric band 12 will be reduced once the obstruction 64 passes through the constriction of the gastric lumen 33. The fluid pressure in the reservoir 18 will then exceed the pressure in the inflatable member 24 of the gastric band 12. The pressure differential will cause fluid to flow back from the reservoir 18 to the inflatable member 24 of the gastric band 12, until the pressure between the inflatable member 24 and the reservoir 18 is again equalized. The fluid returning from the reservoir 18 to the inflatable member 24 can not pass through the one-way valve 52. Instead, the fluid will return to the inflatable member 24 through the flow control valve 54, at a slow flow rate.

In one embodiment, the reservoir 18 may be configured to exert a compressive, or elastic, force against the fluid contained within the reservoir 18. The force may further contribute to the pressure of the reservoir 18.

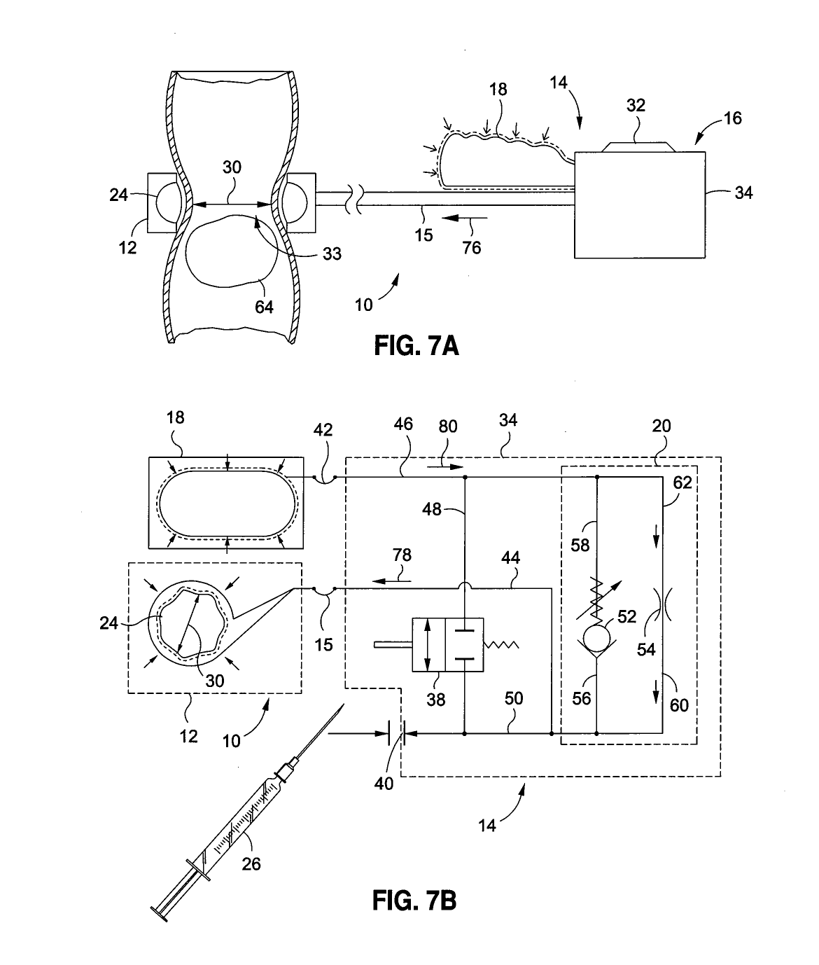

FIG. 7A illustrates a schematic representation of the state of the gastric banding system 10 after the obstruction 64 has passed through the portion of the patient's gastric lumen 33 being constricted. A short arrow 76 indicates fluid flow at a slow rate from the reservoir 18 to the inflatable member 24 of the gastric band 12. The reservoir 18 slowly decreases in size due to fluid exiting the reservoir 18 at a slow rate.

FIG. 7B illustrates a schematic view of the gastric banding system 10 in the state shown in FIG. 7A. The diameter 30 of the inflatable member 24 of the gastric band 12 is shown to slowly decrease. The inflatable member 24 slowly returns to the constricted state shown in FIGS. 5A and 5B. A short arrow 78 indicates fluid flowing at a slow rate from the flow control valve 54 to the inflatable member 24 of the gastric band 12. A short arrow 80 indicates fluid flowing at a slow rate from the reservoir 18 to the flow control valve 54.

The slow flow rate of fluid from the flow control valve 54 to the inflatable member 24 beneficially constricts the gastric lumen 33 slowly after the obstruction 64 has passed. The slow return reduces the possibility of damage to the patient's stomach that could be caused by a relatively quick return of fluid to the inflatable member 24. The slow return of fluid allows the patient's stomach to be slowly compressed, which reduces the possibility of local tissues rupturing or becoming damaged. In addition, wear on the gastric banding system 10 is reduced. Furthermore, the slow return maintains the diameter 30 of the gastric band 12 at an increased size, or the passage state, for a prolonged period of time. It is beneficial to maintain the diameter 30 of the gastric band 12 in the passage state, because it is expected that if one obstruction 64 has passed through the constriction of the gastric lumen 33, then it is likely that another obstruction 64 may be forthcoming. For example, if the patient is eating many large pieces of steak, then it is likely that after one large piece of steak is consumed, then another will be subsequently consumed. The slow return of fluid may keep the gastric band 12 open for a duration that allows for a successive obstruction 64 to more easily pass through the constriction, before the construction closes again. The gastric band 12 will not have to open and close quickly for each successive obstruction 64 passing through the patient's gastric lumen 33.

In one embodiment, the time for the gastric band 12 to open and return to the equilibrium state may be approximately 15 minutes. In one embodiment, the time for the gastric band 12 to open and return to the equilibrium state may be in a range of between approximately 1 minute and 15 minutes. These durations are exemplary in nature and may be varied as desired.

FIG. 8A illustrates a schematic representation of a state of the gastric banding system 10 in which a user, preferably a physician, adjusts the volume of fluid in the inflatable member 24 of the gastric band 12 and the reservoir 18. The adjusted volume of fluid will also adjust the equilibrium pressure of the gastric band 12 and the reservoir 18, as was discussed in relation to FIGS. 5A and 5B.

In the embodiment shown in FIG. 8A, the physician adjusts the volume of fluid in the gastric banding system 10 by inserting a syringe 26 into the access port housing 34, and either adding or removing fluid from the system. FIG. 8A shows fluid being added to the gastric banding system 10. The long arrow 82 indicates fluid flow to the inflatable member 24. The diameter 30 of the gastric band 12 decreases as the size of the inflatable member 24 increases. The size of the reservoir 18 increases.

FIG. 8B illustrates a schematic view of the gastric banding system 10 in the state shown in FIG. 8A. The operation of adjusting the volume of fluid in the gastric banding system 10 is shown to be accomplished through the opening of the shunt valve 38. The shunt valve 38 may comprise a valve capable of being switched between an open position and a closed position. The shunt valve 38 is preferably held in a closed position through a plate and a spring mechanism, which is opened when a force is exerted against the plate by the syringe 26. The spring biases the plate to return the shunt valve 38 to the closed position once the syringe 26 is removed. In the embodiment shown in FIG. 8B, the shunt valve 38 is shown to comprise a valve that may be switched between an open position and a closed position, through a force exerted by the passage of the syringe 26 into the housing 34.

As discussed in relation to FIG. 5B, the shunt valve 38 is directly connected to the reservoir 18 through a fluid conduit 48, and is directly connected to the inflatable member 24 of the gastric band 12 through a fluid conduit 50. The shunt valve 38 is configured to allow fluid to pass to the reservoir 18 and the inflatable member 24 without having to pass through the asymmetric flow regulator 20. The shunt valve 38 is in direct fluid communication with the reservoir 18 and the inflatable member 24, as no valve or control device impedes fluid flow between the shunt valve 38 and the reservoir 18, and the shunt valve 38 and the inflatable member 24. The shunt valve 38 is also in direct fluid communication with the one-way valve 52 and the flow control valve 54.

In one embodiment, the shunt valve 38 may comprise a valve capable of being opened by a fluid pressure exerted against the valve 38, by fluid exiting or entering the syringe 26. In one embodiment, the shunt valve 38 may comprise any valve capable of producing equivalent operation, capable of opening and closing to allow fluid to flow to the reservoir 18 and the inflatable member 24, without having to pass through the asymmetric flow regulator 20.

The operation of the shunt valve 38 is schematically represented in FIG. 8B as the valve element, indicated by the reference number 38, and a filling point 40. The valve element indicated by reference number 38 represents the valve feature of the shunt valve 38, and the filling point 40 represents the ability of the shunt valve 38 to receive and withdraw fluid from the syringe 26.

FIG. 8B illustrates a state of the system 10 in which the shunt valve 38 is open, and fluid is able to flow to the inflatable member 24 and the reservoir 18. A long arrow 84 represents a rapid flow of fluid, or fast flow rate, to the inflatable member 24 through the fluid conduit 44. A long arrow 86 represents a fast flow rate to the reservoir 18 through the fluid conduit 46. The inflatable member 24 inflates with fluid, reducing the diameter of the gastric band 12. Further, the reservoir 18 inflates with fluid. The fast flow rate may be defined as a rate being faster than fluid may pass through the flow restriction device, which is shown in FIG. 8B to comprise a flow control valve 54.

FIG. 9 illustrates a cross-sectional view of one embodiment of the access port 16, illustrating the shunt valve 38. The access port 16 includes a housing 34, a septum 32, and a fluid chamber 36 contained within the housing 34. The septum 32 covers the fluid chamber 36, to prevent fluid from leaving the chamber 36. The septum 32 is preferably needle penetrable and self sealing, and is made from a material such as silicone.

The shunt valve 38 is integrated with the fluid chamber 36. The shunt valve 38 includes a plate 88 and a spring 90 that biases the plate 88 in a direction towards the septum 32. The plate 88 is biased to block passage of fluid from the fluid conduit 50 leading to the inflatable member 24 (shown in FIG. 8B), to the fluid conduit 48 leading to the reservoir 18 (shown in FIG. 8B). The closed fluid passage between the inflatable member 24 and the reservoir 18 allows fluid to only travel through the asymmetric flow regulator 20.

FIG. 10 is a cross-sectional view of the access port 16 shown in FIG. 9, after a syringe 26 needle has penetrated the septum 32 and contacted the plate 88. FIG. 10 also represents the state of the access port 16 shown schematically in FIG. 8B. The force of the syringe 26 needle against the plate 88 causes the spring 90 to compress, and opens up a fluid channel 94 leading to the reservoir 18, and opens up a fluid channel 92 leading to the inflatable member 24. Once the valve 38 opens, the fluid within the fluid chamber 36 equalizes pressure with the fluid contained in the reservoir 18 and the inflatable member 24. Fluid may then pass from the syringe 26 to and from the reservoir 18 and the inflatable member 24, through the respective channels 94, 92. The long arrows 84 and 86 represent fluid flow to the respective inflatable member 24 and the reservoir 18. Fluid does not need to pass through the asymmetric flow regulator 20 to reach the reservoir 18 or the inflatable member 24.

After the syringe 26 needle is removed, the shunt valve 38 closes, and the access port 16 returns to the state shown in FIG. 9.

The shunt valve 38 beneficially allows a physician to adjust the fluid level in the reservoir 18 and inflatable member 24, without fluid having to pass through the asymmetric flow regulator 20. For the embodiment of the asymmetric flow regulator 20 shown in FIG. 8B, if fluid passed through the asymmetric flow regulator 20, then it would either need to pass through the one-way valve 52, or the flow control valve 54.

For example, if fluid was entered into the system on the side of the one-way valve 52 that is coupled to the reservoir 18, then the fluid could not pass to the inflatable member 24 through the one-way valve 52. Fluid may only flow through the one-way valve 52 in a direction of: from the gastric band 12 to the reservoir 18. The fluid could only pass slowly through the flow control valve 54. In addition, if fluid was entered into the system on the side of the one-way valve 52 that is coupled to the inflatable member 24, then it could only pass through the one-way valve 52 if the fluid pressure exceeded the threshold pressure of the one-way valve 52. The fluid could otherwise only pass at a slow rate through the flow control valve 54. Thus, without the shunt valve 38, there would be a delayed response between the insertion or removal of fluid through the syringe 26, and the insertion or removal of fluid from the reservoir 18 and/or the inflatable member 24, caused by fluid passing slowly through the flow control valve 54.

The shunt valve 38 also beneficially allows the physician to quickly ascertain the fluid pressures of the reservoir 18 and the inflatable member 24. If the physician uses a syringe 26 having an integrated pressure meter, then the physician may receive more current readings of the fluid pressure in the system 12, than if fluid had to pass through the flow control valve 54 at a slow rate. The shunt valve 38 may lead to more accurate and rapid adjustments of the fluid volumes and pressures in the gastric banding system 10.

The shunt valve 38 also beneficially allows the gastric band 12 to be emptied rapidly without delay. If the gastric band 12 must be quickly removed from the patient's body, the shunt valve 38 may allow the fluid in the gastric band 12 to quickly be extracted if necessary.

The cross sectional view of the access port 16 shown in FIGS. 9 and 10 represent an exemplary configuration of the access port 16. In other embodiments, the asymmetric flow regulator 20 may be positioned in alternative locations throughout the access port housing 34, which produce equivalent results. In addition, the fluid conduits 44, 50, 48, 46 may be routed in any equivalent manner throughout the access port housing 34. The fluid conduits 44, 50, 48, 46 may link to the tube 15 and/or the coupler 42 in a manner to produce equivalent structural and operative configurations as shown, for example, in FIGS. 8A and 8B. In one embodiment, the asymmetric flow regulator 20 may be positioned to a side of the fluid chamber 36. In one embodiment, the size and configuration of the shunt valve 38 may be varied as desired to produce equivalent results. For example, the shunt valve 38 may be structured such that the plate 88 pivots or bends in response to the force exerted by the syringe 26.

In one embodiment, the asymmetric flow regulator 20 may be equivalently replaced by a single valve device. In this embodiment, the single valve device would operate to provide the functions of both the one-way valve 52 and the flow control valve 54, as discussed in relation to FIG. 5B. For example, the single valve device may comprise a valve capable of only allowing a fast flow rate in a first direction when incident fluid pressure exceeds a threshold value. In addition, the single valve device may include a flow restriction device that allows flow at a slow rate in both the first direction, and in a second, opposite direction. The flow restriction device would allow flow from the reservoir to the gastric band. Thus, the single valve device may reproduce the functions of both the one-way valve 52 and the flow control valve 54, yet would be integrated into a single component, unlike the embodiment of the asymmetric flow regulator 20 shown, for example, in FIG. 5B, in which the flow restriction device comprises a device being separate from the valve configured to open when a threshold pressure has been exceeded.

In one embodiment, the flow restriction device may be configured as a device that only allows flow in one direction of: from the reservoir 18 to the inflatable member 24. In this embodiment, the flow restriction device may not allow fluid to pass from the inflatable member 24 to the reservoir 18. The flow restriction device in this embodiment may serve as a return valve, to only allow fluid flow to slowly pass from the reservoir 18 to the inflatable member 24, to return the inflatable member 24 to a constricted state.

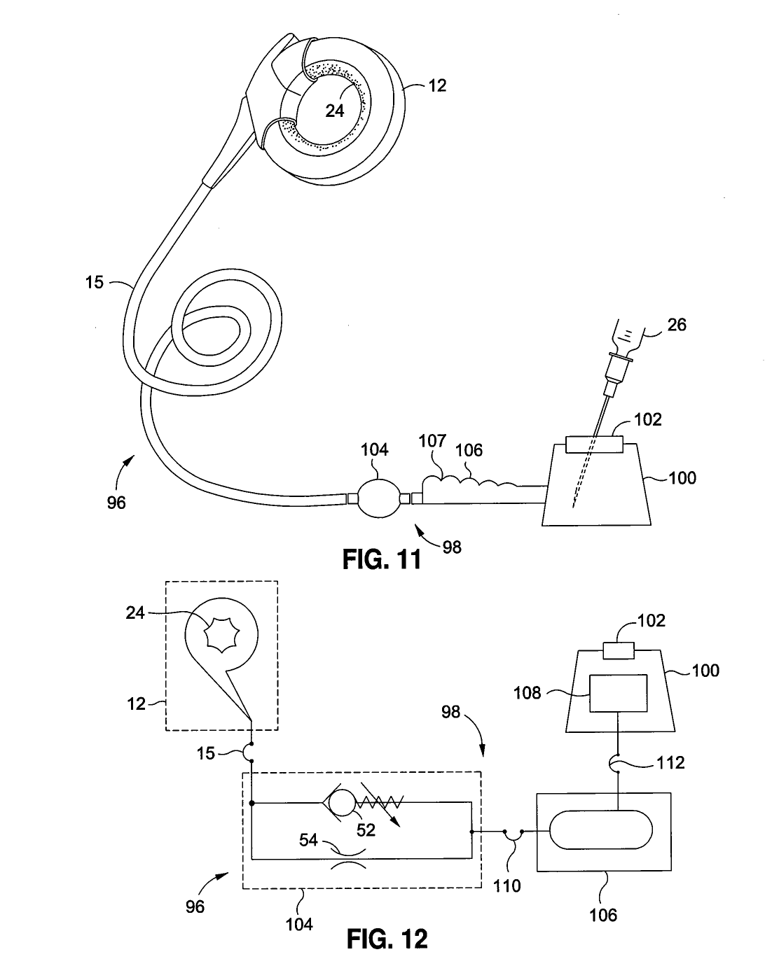

FIG. 11 illustrates an embodiment of a gastric banding system 96 of the present invention, including a gastric band 12, a tube 15, an adjustment system 98 and an access port 100. The access port 100 comprises a standard access port 100 including a septum 102 for receiving the syringe 26. The adjustment system 98 includes an asymmetric flow regulator 104 and a reservoir 106. The reservoir 106 couples to the access port 100 in-line, or in series with the asymmetric flow regulator 104. The tube 15 couples the asymmetric flow regulator 104 to the gastric band 12.

The reservoir 106 is configured similarly as the reservoir 18 shown, for example, in FIGS. 5A and 5B. The reservoir 106 may comprise a flexible balloon, or bladder, capable of expanding or reducing in size in response to a volume of fluid contained within the reservoir 106. The reservoir 106 is positioned outside the access port 100 to allow the reservoir to expand without being limited by the dimensions of the access port 100. One end of the reservoir 106 is coupled to the access port, and the other end is coupled to asymmetric flow regulator 104. The reservoir 106 has two openings, or two inlet/outlets. The reservoir 106 may include a ribbed or ridged structure 107, including a plurality of notches on the surface of the reservoir 106. The notches enhance the flexibility of the reservoir 106 by forming pre-stressed regions of the reservoir 106.

The asymmetric flow regulator 104 is configured similarly as the asymmetric flow regulator 20 shown, for example, in FIG. 5B. However, in this embodiment, the asymmetric flow regulator 104 is not integrated within an access port, but is positioned outside of the access port. One end of the asymmetric flow regulator 104 couples to the reservoir 106 and the other end couples to the tube 15. The access port 100, the reservoir 106, the asymmetric flow regulator 104, the tube 15 and inflatable member 24 of the gastric band 12 are all in serial fluid communication with each other.

FIG. 12 illustrates a schematic representation of the gastric banding system 96 shown in FIG. 11. The asymmetric flow regulator 104 includes the valve, capable of opening when the incident fluid pressure exceeds a threshold value, shown in FIG. 12 as a one-way valve 52, and a flow restriction device, shown in FIG. 12, as a flow control valve 54. The one-way valve 52 and the flow control valve 54, are configured in the manner discussed, for example, in relation to FIG. 5B. The one-way valve 52 is configured to block fluid flow passing from the reservoir 106 to the inflatable member 24, and to only allow fluid to pass from the inflatable member 24 to the reservoir 106 when the incident fluid pressure exceeds a threshold value. The flow control valve 54 is configured to allow fluid flow at a slow flow rate between the reservoir 106 and the inflatable member 24. The one-way valve 52 and the flow control valve 54 may be contained within a shell structure that contains fluid conduits, configured to route fluid in the manner shown in the schematic of FIG. 12.

A coupler 110 links the asymmetric flow regulator 104 to the reservoir 106. A coupler 112 links the reservoir 106 to a fluid chamber 108 of the access port 100. The couplers 110, 112, may be configured to allow components of the gastric banding system 96 to detach from each other. For example, the reservoir 106 may be detachably coupled to the access port 100, and detachably coupled to the asymmetric flow regulator 104. Further, the asymmetric flow regulator 104 may be detachably coupled to the tube 15.

The gastric banding system 96 shown in FIGS. 11 and 12 beneficially allows for a simple design and construction of the adjustment system 98. The access port 100 may comprise a standard access port, which does not require integration of an asymmetric flow regulator 104. The asymmetric flow regulator 104 and reservoir 106 are positioned entirely outside of the access port 100. A standard gastric banding system comprising a band, and a tube coupled to an access port may then be easily modified. For example, referring to a system in which the tube 15 were directly coupled to the access port 100, a user could remove the tube 15 from the access port 100, and then couple the reservoir 106 to the outlet of the access port 100. The user would then couple the asymmetric flow regulator 104 to the reservoir 106. The user would then couple the tube 15 to the asymmetric flow regulator 104 to connect the gastric band 12 to the access port 100. The components of the adjustment system 98 could be removed from the tube 15 and access port 100 by performing these steps in reverse.

The system 96 shown in FIGS. 11 and 12 also beneficially allows for a streamlined, or in-line, position of the reservoir 106, as opposed to the position of the reservoir 18 shown, for example, in FIG. 5A. The streamlined position of the reservoir 106 allows for ease of extraction of the reservoir 106 if the gastric banding system 96 were removed from the patient's body.

A drawback to the embodiment shown in FIGS. 11 and 12 is that a shunt valve, for example the shunt valve 38 shown, for example, in FIG. 8B, may not be easily integrated across the asymmetric flow regulator 104. To adjust the volume of fluid in the system 96, a physician would access the access port 100 by inserting a syringe 26 needle into the fluid chamber 108. If fluid is being introduced into the system 96, then fluid will pass through the reservoir 106, and through the flow control valve 54, before it reaches the inflatable member 24. The restriction caused by the flow control valve 54 may cause a delay effect between the fluid filling the reservoir 106 and the fluid filling the inflatable member 24. The physician may then have to wait for the pressures of the inflatable member 24 and the reservoir 106 to equalize before the physician can determine if the size of the gastric band 12 has been properly adjusted. In addition, if the physician utilizes a syringe having an integrated pressure sensor, then the physician may have to wait, before receiving a proper pressure signal for the gastric banding system 96.

In one embodiment, the reservoir 106 and the access port 100 may be combined as a single unit. The access port 100 may not be necessary for operation of the system 96, if the reservoir 106 is configured to receive a syringe capable of transferring fluid to and from the reservoir 106. In this embodiment, the reservoir 106 may be appropriately fixed to a portion of the patient's body, in a position that is accessible by a syringe.

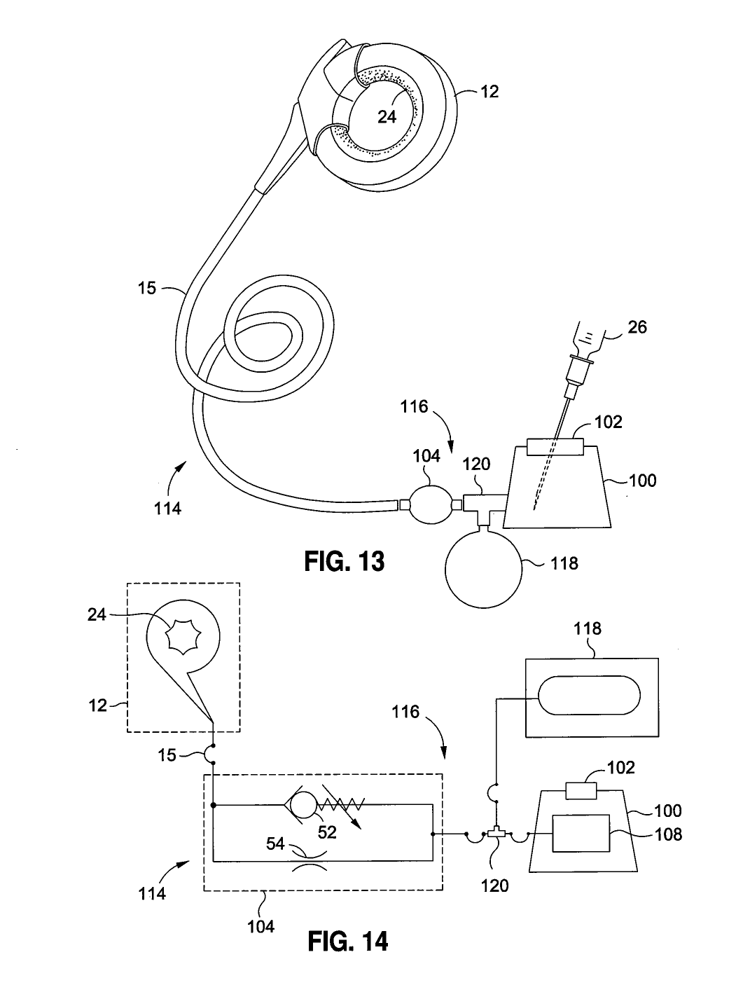

FIG. 13 illustrates an embodiment of a gastric banding system 114 of the present invention including the gastric band 12, the tube 15, an adjustment system 116, and the access port 100. The adjustment system 116 includes the asymmetric flow regulator 104 and a reservoir 118. The reservoir 118 couples to the access port 100 through a t-connector 120, or in parallel with the asymmetric flow regulator 104. The tube 15 couples the asymmetric flow regulator 104 to the gastric band 12.

The reservoir 118 is configured similarly as the reservoir 18 shown, for example, in FIGS. 5A and 5B. The reservoir 118 may comprise a flexible balloon, or bladder, capable of expanding or reducing in size in response to a volume of fluid contained within the reservoir 118. The reservoir 118 may be configured to have only one inlet/outlet, which may reduce the complexity of the reservoir 118, as opposed to a reservoir having two inlet/outlets, as shown, for example, in FIG. 12. The reservoir 118 is positioned outside the access port 100 to allow the reservoir 118 to expand without being limited by the dimensions of the access port 100.

The asymmetric flow regulator 104 is configured similarly as the asymmetric flow regulator 104 shown, for example, in FIGS. 11 and 12. The access port 100, the reservoir 118, the asymmetric flow regulator 104, the tube 15 and inflatable member 24 of the gastric band 12 are all in fluid communication with each other.

FIG. 14 illustrates a schematic representation of the gastric banding system 114 shown in FIG. 13. The asymmetric flow regulator 104 is configured similarly as the asymmetric flow regulator 104 shown in FIG. 12. For example, the one-way valve 52 and flow control valve 54 may be contained within a shell structure, containing fluid conduits configured to route fluid in the manner shown in the schematic of FIG. 14.

The t-connector 120 couples the asymmetric flow regulator 104 and the access port 100 to the reservoir 118. The t-connector 120 may be configured to allow the asymmetric flow regulator 104 and the access port 100 and the reservoir 118 to detach from each other.

The gastric banding system 114 shown in FIGS. 13 and 14 operates similarly as the system 96 shown in FIGS. 11 and 12. However, in this embodiment, the reservoir 118 is not linked in series with the asymmetric flow regulator 104 and the access port 100. If fluid is introduced into the access port 100, it does not have to pass through the reservoir 118 before reaching the inflatable member 24. Further, the embodiment allows for a more simplistic reservoir 118 design, which only includes one inlet/outlet, as opposed to the two inlet/outlets of the reservoir 106 shown in FIGS. 11 and 12. The gastric banding system 114 additionally includes the drawback that a shunt valve, for example the shunt valve 38 shown, for example, in FIG. 8B, may not be easily integrated across the asymmetric flow regulator 104.

The embodiments of gastric banding systems, and adjustment systems shown throughout this application are not limited to gastric systems, or systems for the treatment of obesity. The gastric banding systems and adjustment systems may be integrated into any medical device wherein an inflatable cuff is desired to quickly open in response to an obstruction or large impulse force being applied to the cuff.

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for constricting a stomach of a patient for treating obesity, the system comprising:
a gastric band having an inflatable member configured to contain fluid and apply constriction to a portion of a gastric lumen of the stomach, the inflatable member being moveable from a constricted state to a passage state for allowing an obstruction to pass through the portion of the gastric lumen, the passage state being when less fluid is contained in the inflatable member than in the constricted state;
a valve configured to move from a closed position to an open position when a pressure of the fluid from the inflatable member increases over a threshold in response to the obstruction passing through the gastric lumen, and to allow the fluid from the inflatable member to pass through the valve at a first flow rate;
a reservoir configured to receive the fluid passed through the valve, allowing the inflatable member to move from the constricted state to the passage state, and allowing the obstruction to pass through the portion of the gastric lumen; and
a flow restriction device configured to allow the fluid received by the reservoir to pass from the reservoir to the inflatable member at a second flow rate that is less than the first flow rate, allowing the inflatable member to return to the constricted state.

2. The system of claim 1 wherein the flow restriction device comprises a device being separate from the valve.

3. The system of claim 1 wherein the flow restriction device is configured to allow fluid from the inflatable member to pass to the reservoir.

4. The system of claim 1 wherein the valve is a one-way valve configured to only allow fluid to pass through the one-way valve in a flow direction of: from the inflatable member to the reservoir.

5. The system of claim 1 wherein the threshold is defined by a structural configuration of the valve.

6. The system of claim 5 wherein the threshold is variable.

7. The system of claim 1 wherein the flow restriction device is a flow control valve.

8. The system of claim 1 wherein the flow restriction device is configured such that the second flow rate is variable.

9. The system of claim 1 wherein the valve and the flow restriction device are in fluid communication with the reservoir in a parallel configuration.

10. The system of claim 1 wherein the valve and the flow restriction device are in direct fluid communication with the reservoir.

11. The system of claim 1 wherein the valve is in direct fluid communication with the flow restriction device.

12. The system of claim 1 wherein the reservoir is configured such that a volume of the reservoir increases automatically when the reservoir receives the fluid passed through the valve.

13. The system of claim 12 wherein the reservoir comprises a flexible bladder.

14. The system of claim 1 wherein the valve is configured to open automatically when a pressure of the fluid from the inflatable member exceeds the threshold.

15. The system of claim 1 wherein the gastric band is configured to encircle the portion of the gastric lumen of the stomach to form a diameter, the diameter having a first size when the inflatable member is in the passage state, and having a second size when the inflatable member is in the constricted state, the first size being larger than the second size.

16. The system of claim 1 further comprising an access port, wherein the valve and the flow restriction device are positioned within the access port.

17. The system of claim 16 further comprising a shunt valve configured to open to allow fluid to pass to the reservoir and to the inflatable member without passing through the valve and without passing through the flow restriction device.

18. The system of claim 17 wherein the shunt valve is in direct fluid communication with the reservoir.

19. The system of claim 1 wherein the obstruction is a bolus of food.

20. A system for constricting a stomach of a patient for treating obesity, the system comprising:
a gastric band having an inflatable member configured to contain fluid and apply constriction to a portion of a gastric lumen of the stomach, the inflatable member being moveable from a constricted state to a passage state for allowing an obstruction to pass through the portion of the gastric lumen, the passage state being when less fluid is contained in the inflatable member than in the constricted state;
a valve configured to move from a closed position to an open position when a pressure of the fluid in the inflatable member increases over a threshold in response to the obstruction passing through the gastric lumen, the valve in the open position allowing fluid to pass through the valve at a first flow rate;
a reservoir configured to receive fluid when the valve is in the open position, causing the inflatable member to move from the constricted state to the passage state, and allowing the obstruction to pass through the portion of the gastric lumen; and
a flow restriction device configured to pass fluid through the flow restriction device at a second flow rate that is less than the first flow rate, allowing the inflatable member to return to the constricted state from the passage state.
